(19) United States
(12) Patent Application Publication
(10) Pub. No.: US 2026/0293379 A1
(43) Pub. Date: Sep. 24, 2026

(54) OPTOELECTRONIC SUBSTRATE AND LIGHT SOURCE AND METHOD OF MANUFACTURING THEREOF

(71) Applicant: Ingantec Corporation, Milwaukee, WI (US)

(72) Inventors: Alan S. Yeung, Milwaukee, WI (US); Vincent Rienzi, Goleta, CA (US); David J. Hemke, San Jose, CA (US)

(73) Assignee: Ingantec Corporation, Milwaukee, WI (US)

(21) Appl. No.: 19/547,368

(22) Filed: Feb. 23, 2026

Related U.S. Application Data
(60) Provisional application No. 63/762,194, filed on Feb. 24, 2025.

(51) Int. Cl. H10H 20/825; A61B 3/10; A61B 3/14; H10H 20/01; H10H 20/812; H10H 20/815

(52) U.S. Cl. CPC H10H 20/8252; H10H 20/01335; H10H 20/812; H10H 20/815; A61B 3/102; A61B 3/14

(57) ABSTRACT

Disclosed herein is an optoelectronic structure that includes at least two light-emitting diode (LED) subpixels, laterally or vertically arranged in a substantially common plane, wherein at least one LED subpixel emits light at a wavelength different from at least one other subpixel, and wherein the optoelectronic structure further includes at least a buffer layer, a sublayer, or both, as well as an n-doped III-nitride layer, a light emitting active region, and a p-doped III-nitride layer, wherein at least one of the at least two LED subpixels includes either a strain-relaxed buffer layer or strain-relaxed sublayer, or an enhanced p-doped III-nitride layer modified via dopant concentration adjustments, material composition changes, a porous structure, tunnel junction, or any combination thereof.

### CROSS-REFERENCE TO RELATED APPLICATION

This application claims priority to U.S. Provisional Patent Appl. No. 63/762,194 filed on Feb. 24, 2025, the disclosure of which is incorporated herein by reference in entirety for all purposes.

### FIELD OF THE INVENTION

This invention relates generally to light emitting diode (LED) technology, and more particularly to micro-LEDs as miniature dual or multiple band light sources with modulation linewidth and tunable wavelength or waveform having efficient spectral emission and low-loss optical interconnect.

### BACKGROUND OF THE INVENTION

Optical Coherence Tomography (OCT) is a powerful, non-invasive biomedical imaging technique that uses low-coherence interferometry to generate high-resolution, cross-sectional, and three-dimensional images of biological tissues and microstructures at micrometer-scale axial and lateral resolution (typically 1-10 μm axially). Since its introduction in the early 1990s, OCT has revolutionized clinical diagnostics, particularly in ophthalmology, where it has become the gold standard for retinal and optic nerve assessment. Its speed, repeatability, and quantitative capabilities enable precise monitoring of conditions such as diabetic macular edema (DME), glaucoma (via retinal nerve fiber layer thickness and optic nerve head evaluation), age-related macular degeneration (AMD), and inherited retinal dystrophies. In complementing and/or replacing invasive procedures like fluorescein angiography, OCT supports early detection, progression tracking, and treatment guidance in routine eye clinic settings.

Traditional ophthalmic OCT systems employ tabletop or desktop setups with fixed chin rests and optics optimized for external imaging through the transparent ocular media (cornea and lens). These systems typically use single-wavelength, light sources—such as super luminescent diodes (SLDs), swept-source lasers, or diode-pumped solid-state (DPSS) lasers—centered in the near-infrared (~800-1300 nm) region of the light spectrum for deep tissue penetration and high axial resolution via short coherence lengths. Backed by decades of clinical data, these SLD and DPSS sources contribute to advances in patient care and diagnostics, yet they result in bulky, stationary, and high-cost OCT instruments that have limited accessibility beyond specialized settings.

To expand OCT applications beyond ocular imaging—such as intravascular OCT for coronary arteries, endoscopic OCT for colon or bile ducts, or handheld probes for surface tissues—significant adaptations and upgrades are required. These include miniaturized, flexible, sterile probes for insertion or close contact, modified scanning mechanisms (e.g., from stationary to maneuverable catheters or handheld heads), and enhanced light delivery and collection via single-mode optical fibers. These adaptations enable high-resolution “optical biopsy” but would require higher electroluminescence and efficient emission of visible light and lower loss in optical interconnect. For instance, intravascular OCT and endoscopic applications require the use of a single-mode optical fiber to transmit light to the tissue and collect backscattered signals by insertion into coronary arteries and for imaging the colon or bile ducts to detect inflammation or early cancers.

Handheld or portable OCT scanning heads, connected by fiber-optic cables to a central light engine, may offer an upgrade to traditional OCT equipment, thus allowing the clinician to manually position and scan directly on or near the tissue surface. This approach is favored to expand OCT adoption and enable increasing usage in clinics, operating rooms, or primary care facilities.

Besides the need to replace SLDs and laser diodes with lower cost, compact light sources for improving accessibility and portability, the light source properties are important design factors. Coherence property of a light source plays an essential role in ensuring image quality and high resolution in OCT and other opto-medical diagnostics tools. A light source's coherence property can be characterized by its temporal coherence and spatial coherence values. Temporal (related to spectral bandwidth) and spatial (related to beam uniformity) coherence properties critically influence OCT image quality, resolution, and artifacts. High temporal coherence (narrow linewidth) enables fine axial resolution but can introduce speckle noise (granular interference patterns) that degrade contrast. High spatial coherence may improve image sharpness but exacerbate speckle.

Conventional broadband sources like white lamps or standard LEDs offer low temporal coherence (reducing speckle) but suffer from low spatial coherence which can greatly reduce the sharpness of the images. Lasers provide excellent spatial coherence for sharp images, but high temporal coherence could lead to pronounced speckle. While speckle is often mitigated by techniques like time-averaged superposition of phase-randomized images, these techniques often come at the cost of increased system complexity. They tend to raise the computation costs and decrease the bandwidth of the reconstructed images.

To facilitate higher image sharpness with reduced speckle, light sources with high spatial coherence and low temporal coherence are desired. When compared with LED and mini-LEDs, micro-LEDs are ideal as OCT light sources if the size of the LED pixels can be reduced to below 100 micrometers or microns (μm). These sub-100 μm micro-LED semiconductor emitters can be arranged in dense arrays and have emerged as promising alternatives to existing light sources for OCT and related opto-medical tools. When engineered appropriately, micro-LEDs may offer a balance of high spatial coherence (for sharpness) and low temporal coherence (for reduced speckle). A further enhancement is achievable when two or more emission bands with low temporal coherence across the light spectrum are provided in a broadband or multi-band emission mode. Key advantages include (a) array integration for dual-or multi-wavelength operation, e.g., visible-light OCT at 400-700 nm for enhanced superficial tissue contrast via stronger scattering and absorption by chromophores like hemoglobin; (b) high brightness, efficiency, and low power consumption, supporting compact, battery-powered, or portable systems; (c) array flexibility for structured illumination, e.g., full-field or multi-spot excitation, to simplify optics in full-field time-domain OCT or spectroscopic imaging; and (d) compact integration into probes, endoscopes, or other devices, reducing system size and complexity.

To enable full-field OCT multicolor imaging and vis-OCT applications, system engineers may explore monolithic Red, Green and Blue (RGB) integration of micro-LEDs to realize multi-band micro-LED sources. While blue and green micro-LEDs are generally available in the 5-10 μm range, red micro-LEDs remains challenging at such sub-10 μm level, utilizing two different LED optoelectronic materials—AlInGaP (III-phosphides) and InGaN (III-nitrides). Micro-LED designs based on red AlInGaP emission are hampered by diminishing quantum efficiency of phosphides at smaller pixel sizes at or below around 20 μm. While the size-dependent efficiency drop of AlInGaP micro-LEDs may be remedied by sidewall passivation, thermal stability issues persist.

InGaN-based red micro-LEDs are emerging as viable light sources due to their compatibility with their blue and green InGaN counterparts, enabling monolithic RGB integration and better size-independent performance. For InGaN based micro-LEDs, however, dual-or multi-band micro-LED sources—particularly in the red spectral region—remains challenging due to the “green gap” problem in InGaN-based LEDs, where high indium content for longer wavelengths may lead to lower quantum efficiency as a result of strain-induced defects and quantum-confined Stark effect (QCSE) issues. QCSE is a phenomenon that affects the efficiency of light-emitting devices, particularly in InGaN/GaN quantum wells. Quantum-dot technologies have been utilized to leverage efficient blue micro-LED emission to resolve such issues. However, this color conversion approach faces directional emission limitations and is generally undesirable for providing focused beams that are required for low-loss data or imaging interconnect.

Achieving sufficient electroluminescence, low temporal coherence across bands, and high spatial coherence are therefore required to make III-nitride based micro-LEDs possible for vis-OCT usage. This requires advanced and inventive approaches in epitaxial processes, e.g., V-defect engineering, strain relaxation, or optimized quantum wells.

III-nitride semiconductors—commonly referred to as “III-N,” “nitride-based,” “nitride,” or similar terms—encompass a broad family of materials with the general with alloy compositions AlₓIn_{y}Ga_{z}N, where 0≤x≤1, 0≤y ≤1 , 0≤z≤1, and x+y+z=1, enabling tunable bandgaps from ultraviolet (AlN) through visible (GaN, InGaN) to near-infrared (high-In InGaN). These materials have driven major advances in optoelectronics, particularly blue, green, and emerging red LEDs.

It is to be understood that this invention disclosure is not limited to the traditional definition of III-nitride, but includes the rest of group 13 (previously called “IIIA” or simply “III”) elements, Boron (B) and Thallium (Th), as well as those of group 3 (previously called “IIIB”), including scandium (Sc), Yttrium (Y), and Lanthanum (La), such that the layers may comprise any alloy composition of the (La, Y, Sc, Ga, Al, In, Th, B)N semiconductors having the formula LaₛYₜScᵤGaᵥAl_{w}InₓTh_{y}B_{z}N where 0≤s≤1, 0≤t≤1, 0≤u≤1, 0≤v≤1, 0≤w≤1, 0≤x≤1, 0≤y≤1, 0≤z≤1, and s+t+u+v+w+x+y+z=1. Furthermore, other impurity dopant atoms, such as Magnesium (Mg), Silicon (Si), Carbon (C), Germanium (Ge), or other atoms may be included within the LED epitaxial structure without deviating from the scope of the invention.

III-nitride LEDs have the wurtzite crystal structure, which has several different crystallographic orientations. The most common is the metal polar c-plane (0001) and is the preferred embodiment, but any other crystallographic orientation or combination thereof is within the scope of the present invention, including Nitrogen polar c-plane (0001̅), a nonpolar plane, such as an a-plane or m-plane, or a semipolar plane, such as (101̅1), (202̅1), (202̅1̅), or (112̅2).

Conventional III-N semiconductors are grown planar on the wurtzite metal polar c-plane. For visible III-N LEDs, there exists a large total polarization discontinuity at the quantum well (QW) (i.e. InGaN) and quantum barrier (i.e. AlGaN) interface. This arises from spontaneous and piezoelectric polarization, generating strong internal electric fields (~MV/cm) that tilt bands and spatially separate electrons and holes via quantum confined Stark effect (QCSE). This leads to large internal electric fields in the QWs. The piezoelectric contribution to the total polarization discontinuity is exacerbated as indium composition in the QW increases, which is required for longer wavelengths, and more optically red colors. Consequently, internal quantum efficiency (IQE) declines sharply toward red emission—the core of the “green gap” problem being extended to red. This increased discontinuity leads to widening energetic barriers in the LED energy band profiles, hindering carrier injection into QWs, and reducing radiative recombination of electrons and holes in the QWs. As a result, the micro-LED's internal quantum efficiency (IQE) decreases as the emission spectra goes to longer wavelengths. To mitigate these effects, engineers are exploring various approaches including V-defect and nanoporous strain engineering.

V-defects (V-shaped pits or V-pits) are inverted pyramidal defects originating from threading dislocations and are a type of crystal defect. However, V-defect engineering can be employed to improve long-wavelength (i.e. green and red) III-N LED performance, namely increased peak wall-plug efficiency (WPE) and reducing the severity of the green gap challenge. In general, defects disrupt the ordering and purity of crystals; yet V-defects may play a pivotal role not only in reducing the energetic barriers that holes and electrons must overcome to radiatively recombine, but also in the state of strain relaxation of the semiconductor alloys of the LED.

V-defect sidewalls are {101̅1} semipolar planes, and could significantly reduce the energy discontinuity that carriers encounter for radiative recombination via lateral injection through the semipolar sidewalls into the c-plane QWs rather than vertical c-plane transport. Their sidewalls expose {10 11} semipolar facets, creating lower-energy pathways for carrier transport compared to polar c-plane barriers. Holes (lower mobility, higher barriers in c-plane) preferentially inject laterally via semipolar sidewalls, bypassing polarization-induced vertical barriers and enabling more uniform carrier distribution in QWs. This can enable higher indium composition within the quantum wells, engendering better efficiency, engineered emission polarization, other benefits, or any combination thereof. V-defects facilitate localized higher carrier density near sidewalls, allowing better QCSE screening. through polarization engineering and self-screening. Specifically, techniques in polarization engineering, carried out in crystal growth in a metalorganic chemical vapor deposition (MOCVD) reactor, may include strain control and growth direction control to mitigate the polarization-induced electric field in QWs and partially relax compressive strain in high-In QWs. This could lead to reducing piezoelectric fields and enabling higher indium incorporation for true red emission.

For III-N LEDs grown in a MOCVD reactor, the preferred growth conditions for single crystal thin films of the binary AlN, GaN, and InN semiconductors vary significantly, with AlN often being grown around 1400° C. and InN being grown closer to 500° C. As such, when growing heterostructures, compromises must be made from one layer to the next, depending on targeted alloy composition. Often, LED active regions use relatively cold temperatures when growing the InGaN QWs to increase indium composition, but growing GaN or AlGaN QBs at such low temperatures would lead to defect formation and worse crystal quality. As such, these subsequent layers typically are grown hotter, to the detriment of the InGaN QWs. Indium will desorb if temperatures are too high. The indium that remains in the QWs is effectively annealed during the QB and p-side growth when exposed to elevated temperatures for sustained periods of time. This leads to reduced LED IQE, with longer growth times leading to lower IQEs. These challenges limit high-indium red QWs, underscoring the need for optimized growth sequences, strain-relief layers (e.g., superlattices initiating V-defects), or alternative techniques to balance crystal quality, optimize indium retention, and boost device performance.

It is thus desirable to have improved products and processes that would fundamentally address the key barriers preventing widespread clinical adoption of vis-OCT: bulky/expensive light sources, limited portability, insufficient bandwidth/spectral control for multi-wavelength or spectroscopic imaging, and challenges in achieving high spatial coherence with low temporal coherence (to minimize speckle while maintaining sharpness and sub-micron axial resolution). Current vis-OCT systems primarily rely on expensive, complex broadband sources like supercontinuum lasers, Ti: sapphire-pumped photonic fibers, swept-sources with second-harmonic generation, or combined SLDs—often limiting them to research labs or high-end clinical setups. Proof-of-concept work has demonstrated micro-LED arrays as illumination sources in full-field time-domain vis-OCT for spectroscopic imaging, but these remain early-stage with severe limitations in coherence properties, power, bandwidth, and red-region performance.

It is therefore desirable to produce a monolithic, broadband and/or tunable multi-band InGaN micro-LED arrays optimized for vis-OCT, and to produce suitable dual-or multi-band light sources based on InGaN optoelectronic structures that have low temporal coherence and high spatial coherence. One of several exemplary objects of the invention is to construct an optoelectronic structure that serves as a dual band micro-LED light source with micro-LED pixels-so that OCT images can be more accurately taken to help elucidate tissue morphology with increased resolution and contrast (e.g., without speckle, etc.). Various exemplary biomedical applications, such as bench top and handheld devices used for imaging and diagnosis in endoscopy and ophthalmology may also benefit from such improvements. Note these devices may use battery and/or AC power and because of different wavelengths, pulse mode, and relative quantum efficiency of these micro-LED light sources, more efficient microstructure, fiber optics systems, optical interconnects, and other integrated/hardware components are likely needed.

### BRIEF SUMMARY

In at least some embodiments, the invention is directed to a dense, integrated micro-LED array, with pixel sizes <50-100 μm, using advanced InGaN-based epitaxy to deliver broad spectral coverage across the visible range (e.g., 440-680 nm or wider, including strong red emission) from a single monolithic chip or tightly integrated RGB/multi-band array, providing the short coherence length needed for ultrahigh axial resolution (~0.6-1.3 μm in tissue, far superior to NIR-OCT's 5-10 μm). The micro-LED based light source, or light engine, provides low temporal coherence (broad effective bandwidth per band or across the array) combined with high spatial coherence (via array design or meta surface integration) to drastically reduce speckle noise without computational averaging techniques that slow imaging or increase complexity. By deploying nanoporous strain engineering, V-defect engineering, superlattice strain relief, ALD passivation, semipolar orientations, single/graded and ultrathin quantum wells, and meta surfaces for directionality/efficiency boosts, this invention enables low-power, compact sources suitable for battery-powered handheld or endoscopic probes. The result is a compact, chip-scale light source supporting structured or full-field illumination via the use of micro-LED arrays that could provide programmable multi-spot, line-field, or full-field excitation, eliminating mechanical scanning in some configurations (e.g., full-field vis-OCT) and supporting dual-modal (OCT and autofluorescence or oximetry) or spectroscopic vis-OCT natively.

In at least some other embodiments, the invention is directed to an optoelectronic structure comprising at least two light emitting diode (LED) subpixels, laterally or vertically arranged in a substantially common plane, wherein at least one LED subpixel emits light at a wavelength different from at least one other subpixel, and wherein the optoelectronic structure further includes at least: a buffer layer, a sublayer, or both; an n-doped III-N layer; a light emitting active region; and a p-doped III-N layer, wherein at least one of the at least two LED subpixels includes either a strain-relaxed buffer layer or strain-relaxed sublayer, and/or an enhanced p-doped III-N layer modified via dopant concentration adjustments, material composition changes, a porous structure, tunnel junction, or any combination thereof.

In at least some other embodiments, the invention relates to a method of manufacturing micro-LED subpixels that include the at least two light emitting diode (LED) subpixels as described herein, wherein the at least two LED subpixels are laterally or vertically arranged and monolithically fabricated on a substantially common plane on a structure, and wherein the two or more light bands are emitted without requiring pick-and-place or mechanical transfer of subpixels.

In at least some other embodiments, the invention is directed to an optoelectronic structure that includes a first subpixel that emits light at a first wavelength, and comprises; at least one of a first buffer layer and a first sublayer; a first n-doped III-N layer; a first light emitting active region; and a first p-doped III-N layer; and a second subpixel that emits light at a second wavelength that is different that the first wavelength, and comprises; at least one of a second buffer layer and a second sublayer; a second n-doped III-N layer; a second light emitting active region; and a second p-doped III-N layer; wherein the first and second subpixels are laterally arranged on a substantially common plane, and wherein at least one of the first buffer layer, first sublayer, second buffer layer, and second sublayer is either strain relaxed or is an enhanced p-doped III-N layer modified via at least one of dopant concentration adjustments, material composition changes, ultrathin sublayers, tunnel junction(s), and a porous structure.

In at least some other embodiments, the invention is directed to a light source comprising at least two micro-LED subpixels whose peak wavelengths differ by at least 30 nm from each other and are configured for use in Optical Coherence Tomography, holographic displays, endoscopy, ophthalmology, and/or other biomedical imaging and diagnosis tools to discern body tissue morphology, cell structures, blood cell existence, and/or vascular dynamics within blood vessels and/or structures within the retina or other human organs and tissues.

In at least some other embodiments, the invention is directed to a micro-LED device comprising: a first LED subpixel that emits light at a first wavelength, and comprises: at least one of a first buffer layer and a first sublayer; a first n-doped III-nitride layer; a first light emitting active region; and a first p-doped III-nitride layer; and a second LED subpixel that emits light at a second wavelength that is different than the first wavelength, and comprises: at least one of a second buffer layer and a second sublayer; a second n-doped III-nitride layer; a second light emitting active region; and a second p-doped III-nitride layer; wherein the first and second LED subpixels are laterally, vertically, or any combination thereof arranged on a substantially common plane, and wherein at least one of the first buffer layer, first sublayer, second buffer layer, and second sublayer is either strain-relaxed or is an enhanced p-doped III-nitride layer modified via at least one of dopant concentration adjustments, material composition changes, and a porous structure.

In at least some other embodiments, the invention is directed to a micro-LED device comprising an optical imaging device that includes multiple LED subpixels that are laterally, vertically, or any combination thereof arranged and monolithically fabricated on a substantially common plane on a structure without requiring pick-and-place or mechanical transfer of the subpixels as light sources, wherein the micro-LED subpixels are configured to emit in multiple emission spectra and illuminate a target object and to generate a multi-wavelength image of the target object.

In at least some other embodiments, the invention is directed to an Optical Coherence Tomography equipment or device, wherein intravascular OCT and endoscopic applications requiring the use of two or more light bands via an optical fiber to transmit light to the tissue through portable, insertable, or maneuverable forms and collect backscattered signals by insertion into coronary arteries and other intestines or ducts to detect inflammation or early cancers. These adaptations enable high-resolution “optical biopsy” but would require higher electroluminescence and efficient emission of visible light and low loss in optical interconnect.

In at least some other embodiments, the invention is directed to a light source comprising at least two micro-LED subpixels whose peak wavelengths differ by at least 30 nm from each other having waveforms with a full-width at half-maximum (FWHM) of at least 10 nm and are utilized as a light source in a biomedical imaging device to discern at least one of body tissue morphology, cell structures, blood cell existence, vascular dynamics within blood vessels, structures within the retina or tissues within other human organs.

In at least some other embodiments, the invention is directed to a light source for visible-light optical coherence tomography (vis-OCT), comprising: a monolithic micro-LED array comprising a plurality of InGaN-based micro-LED pixels, each pixel having a lateral dimension of less than 100 μm; wherein the micro-LED array is configured to emit light across a broadband visible spectrum including at least blue, green, and red wavelength bands, the emitted light having a low temporal coherence length suitable for achieving axial resolution of 2 μm or less in tissue and high spatial coherence for enhanced image sharpness with reduced speckle noise.

In at least some other embodiments, the invention is directed to an Optical Coherence Tomography (OCT) illumination system, comprising: a micro-LED array light source including a plurality of InGaN-based micro-LED pixels fabricated on a common substrate; drive circuitry coupled to the micro-LED array to selectively activate subsets of pixels for structured illumination; wherein the light source provides broadband emission with an effective coherence length configured to support sub-2 μm axial resolution in vis-OCT while minimizing speckle artifacts without requiring phase-randomization averaging techniques.

Other embodiments, aspects, features, objectives and advantages of the invention will be understood and appreciated upon a full reading of the detailed description and the claims that follow.

### BRIEF DESCRIPTION OF THE DRAWINGS

Embodiments of the invention are disclosed with reference to the accompanying drawings and are for illustrative purposes only. The invention is not limited in its application to the details of construction or the arrangement of the components illustrated in the drawings. The invention is capable of other embodiments or of being practiced or carried out in other various ways. Like reference numerals are used to indicate like components. In the drawings:

FIG. 1 includes a schematic diagram of an exemplary spectrum-domain Optical Coherence Tomography (OCT) system utilizing a tunable dual band light source;

FIG. 2 includes a planar view of an exemplary pair of monolithic RG micro-LED subpixels being combined to form a single micro-LED pixel, and then as a portion of an array on a wafer;

FIG. 3 is a cross-sectional view of an exemplary optoelectronic structure;

FIG. 4A includes a cross-sectional view of the GaN based red micro-LED epitaxy stack of FIG. 3 further illustrating the composition of an exemplary embodiment of the enhanced p-InGaN layer;

FIG. 4B include a cross-sectional view of the GaN based red micro-LED epitaxy stack of FIG. 3 further illustrating the composition of another exemplary embodiment of the enhanced p-InGaN layer;

FIG. 5 is a cross-sectional view of the GaN based red micro-LED epitaxy stack of FIG. 3 illustrating the composition of one exemplary embodiment of the strain-relaxed InGaN sublayer;

FIGS. 6A-6N illustrate exemplary steps of a process (Method A) of fabricating an exemplary optoelectronic structure comprising a monolithic red and green micro-LED;

FIGS. 7A-7K illustrate exemplary steps of another process (Method B) of fabricating an exemplary optoelectronic structure comprising a monolithic red and green micro-LED;

FIGS. 8A-8C illustrate exemplary steps of fabricating and preparing packaged micro-LEDs from an exemplary optoelectronic structure;

FIG. 9 shows an exemplary bench top OCT device comprising a light source based on dual or multi-band micro-LEDs that are fabricated from an exemplary optoelectronic structure, and are equipped with a power supply and fiber optics with efficient light emission and collection via low-loss optical interconnect;

FIG. 10A illustrates an exemplary pen-shaped OCT device comprising a light source incorporating micro-LEDs fabricated from an optoelectronic structure; and

FIG. 10B illustrates an exemplary use of the pen-shaped light source of FIG. 10A for OCT diagnosis of a patient's eye or retina.

### DETAILED DESCRIPTION

Various embodiments of laterally arranged heterogenous micro-LEDs are disclosed herein, but it is to be understood that the present invention is not limited to only lateral arrangements, but includes vertical arrangements or any combination thereof. Common to all these are at least one subpixel that comprises a strain-relaxed sublayer, although in other embodiments, the strain-relaxed sublayer may be omitted. The strain-relaxed sublayer reduces the strain, i.e., provides full or partial strain relaxation, in the subsequently grown epitaxial layers, resulting in changes in lattice constants of the active layers, thus allowing higher quantity of indium incorporation and shifting of the emission spectra to longer wavelength. Strain-relaxed sublayers may include optoelectronic structure comprising nanoporous substrates and/or V defects. Various other exemplary approaches to depositing and fabricating other subpixel elements are also disclosed.

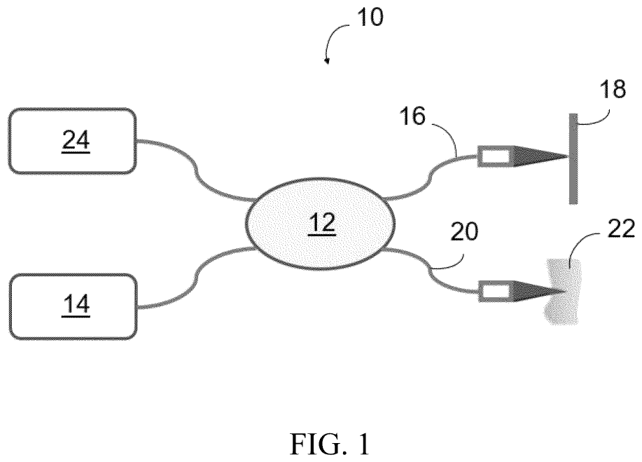

FIG. 1 illustrates an exemplary embodiment of an OCT system 10 having a coupler/beam splitter 12, a display 14, a reference arm 16 directed to a mirror 18, a probed sample arm 20 directed to tissue 22, and a dual band micro-LED light source 24, such as red and green in a scale of 0.1-25 μm, which is ideal for providing high spatial coherence and low temporal coherence. The selection, separation and tuning of the wavelengths of the underlying micro-LED bands can be manipulated to elucidate flow of red and white blood cells within blood vessels, or to discern changes in form and thickness of retinal tissues within the human eyes. In other embodiments, the dual light source 24 can be used to alternate between two wavelengths thus providing convenience and ease of use, as well as a lower cost.

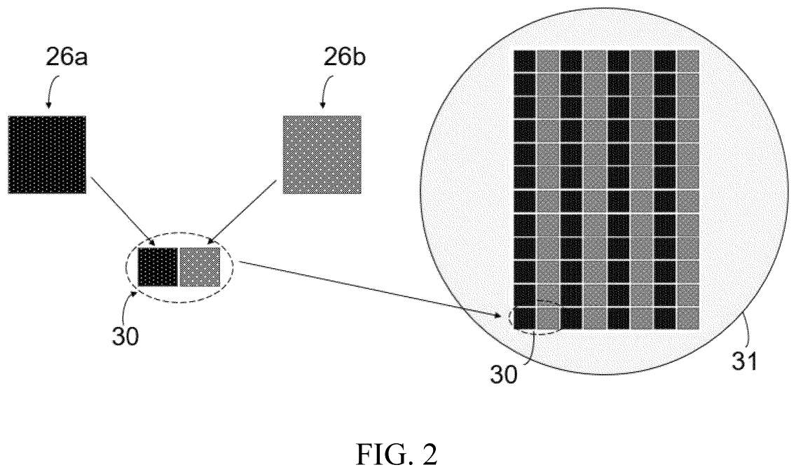

To obtain red and green light bands, or any pair of micro-LED light bands, it is desirable to have micrometer-scale subpixels that can be grown or deposited via Metal-Organic Chemical Vapor Deposition (MOCVD) and similar tools. One exemplary heterogeneous, laterally arranged set of two-color RG subpixels 26 (e.g., 26a for red and 26b for green) is shown in FIG. 2 forming a pixel 30, which further forms a portion of an array of pixels 30 situated on a wafer 31 (e.g., substrate). Here, the exemplary subpixels 26 shown are square; other shapes and forms, such as rectangle, triangle, pentagon, hexagon, concentric, circular, random shapes, etc. and combination thereof, are also disclosed and possible. Arrangements, such as concentric annuli having a common optical axis (a bull's eye pattern), are possible and especially useful for improved compatibility with downstream optics and low-loss optical interconnect and electronics components. Here, red and green (RG) subpixels 26 are shown; however, in at least some embodiments, any combination of two or more subpixels having varying electromagnetic emission characteristics, such as emitting wavelength or waveform, are possible. Although in some embodiments, the subpixels can be the same size, they need not be. There can be gaps between subpixels. These subpixels, with different emission wavelengths or colors, can be directly adjacent to each other, but need not be. A wafer or an application can be constructed with combinations of pixels with different subpixels, for example, there may be more red subpixels in one pixel or area vs another, or there may be fewer red subpixels within another pixel or area vs another, or there may be fewer red subpixels within another pixel or area.

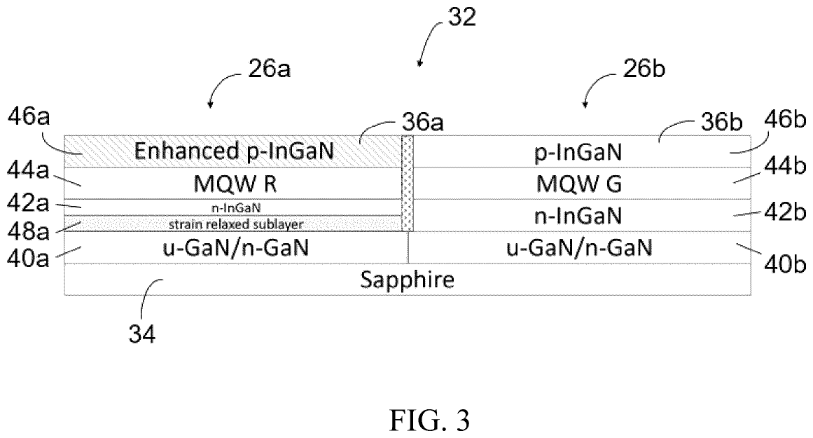

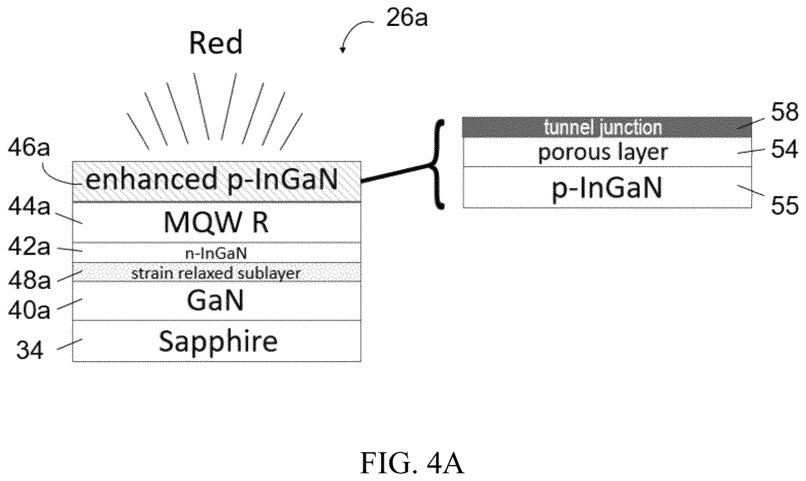

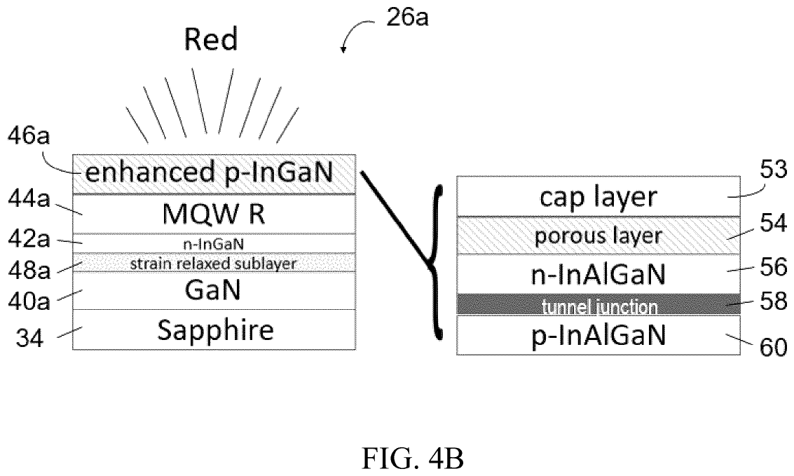

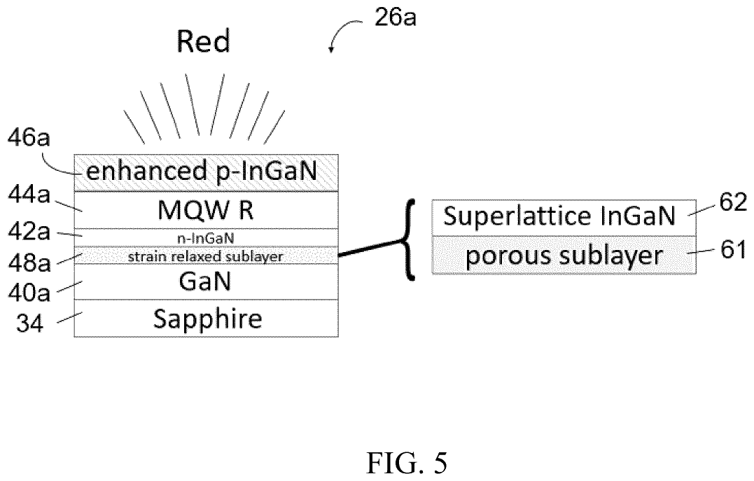

To optimize fabrication and device performance, it is possible and, in some embodiments, particularly useful to have different sizes and shapes of two or more subpixels deposited on the substrate of the light source or device. An exemplary optoelectronic structure 32 is shown in FIG. 3, which can be prepared with a pattern sapphire substrate 34 (e. g,, a sapphire wafer) and red and green subpixels 26a, 26b monolithically deposited in their respective regions. It shall be understood that in at least some embodiments, the sapphire substrate 34 is representative of a base that can include other materials, or be wholly replaced with one or more different materials. Each LED epitaxy stack 36 (e.g., 36a and 36b), be it red, green, or some other wavelength, comprises a buffer layer 40 (e.g., 40a, 40b) of GaN materials, an n-layer 42 (e.g., 42a, 42b), a light emitting active region that is comprised of one or more multi-quantum wells (MQWs) 44 (e.g., 44a, 44b), and a p-layer 46 (e.g., 46a, 46b). The buffer layer may comprise UID GaN and n+GaN (u-GaN/n-GaN), AlGaN, or other GaN-based materials having no or different doping profiles. The red LED epitaxy stack 36a may also have a sublayer 48 (e.g., 48a) wherein it has been modified to provide adequate strain relaxation. The strain-relaxed sublayer may be comprised of multiple layers or sublayers based on InGaN superlattice, half-loop generation/planarization layers, V-defect opening layer, and/or porous GaN or InGaN. This sublayer 48a may be partially or fully strain-relaxed, and is deposited or grown above a buffer layer 40a that comprises u-GaN/n-GaN layers. Within the red LED region, the LED epitaxy stack 36a may be comprised of other layers including but not limited to cap layers and electron blocking layers (EBLs). The p-layer 46a of the red LED epitaxy stack may also be enhanced with different known and later developed methods. In one embodiment, a strain-relaxed sublayer 48a exists under the red subpixel, while a non-porous sublayer may exist under the green subpixel. The differences in lattice constant and degree of strain relaxation due to the existence of this strain-relaxed sublayer account for the different emission spectrum of the red subpixel relative to the green subpixel. Instead of green, a separate blue subpixel can be grown. Likewise, instead of red, an orange or a yellow subpixel can be fabricated. To increase the efficiency and brightness of the device, the p-InGaN layer (or cathode) of the LED can also be optimized by utilizing a graded p-layer via polarization induced process or a graded porous layer, as shown in FIGS. 4A-4B. A doping or porosity gradient may exist within a layer or sublayer over a defined thickness and/or extend across the interface between two layers or sublayers. Likewise, the enhanced p-layer 46a may include a tunnel junction 58 grown on top of a porous layer 54 and p-InGaN layer 55 for performance improvement and stacked (i.e. vertical) monolithic RG or RGB micro-LEDs, or combined vertical/lateral RGB micro-LEDs (see FIG. 4A). In another embodiment, the enhanced p-layer may comprise a cap layer 53, a porous layer 54, an n-InAlGaN layer 56, a tunnel junction 58, and a p-InAlGaN layer 60 (see FIG. 4B). A red shift of emission wavelength and a boost in quantum efficiency are made possible by strain relaxation via various porous substrates and sublayers 61, as well as other strain engineering approaches including the use of superlattice InGaN layers 62, with or without V-defects, as shown in FIG. 5.

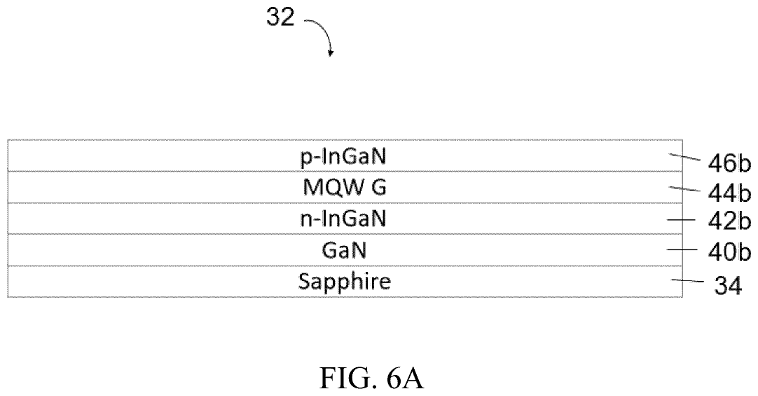

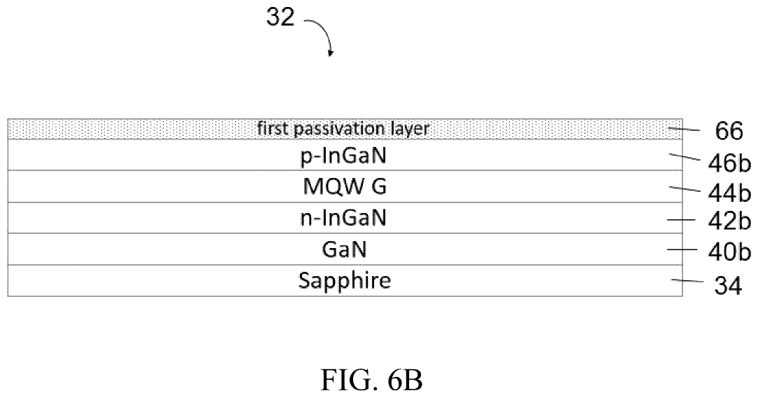

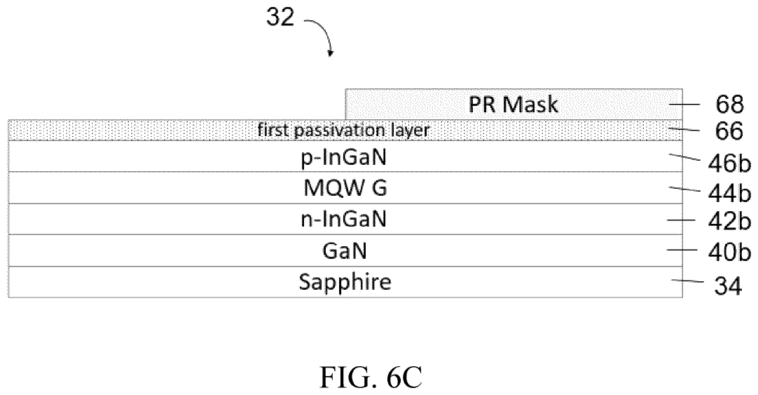

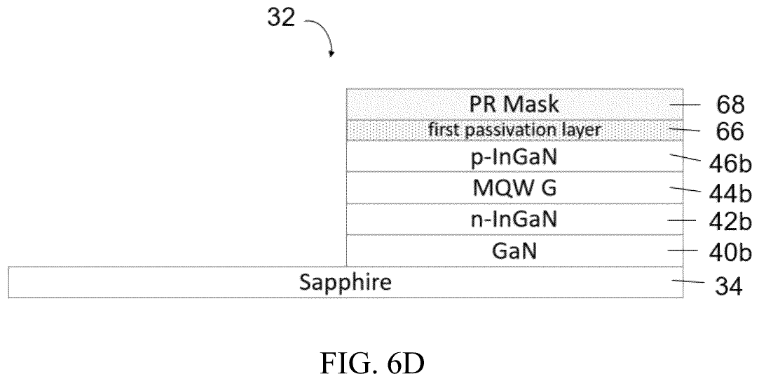

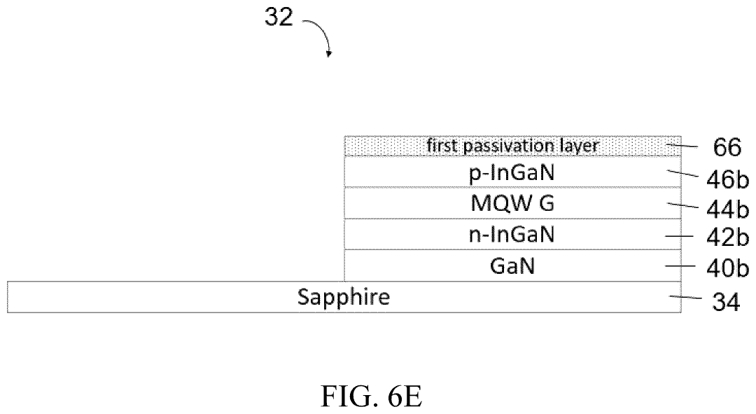

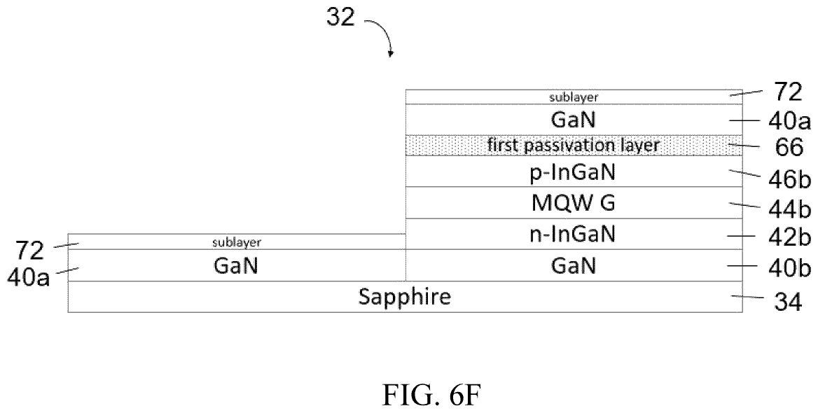

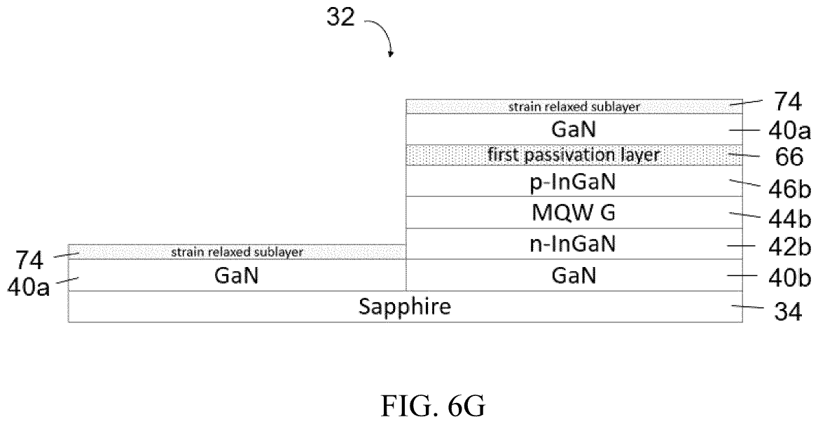

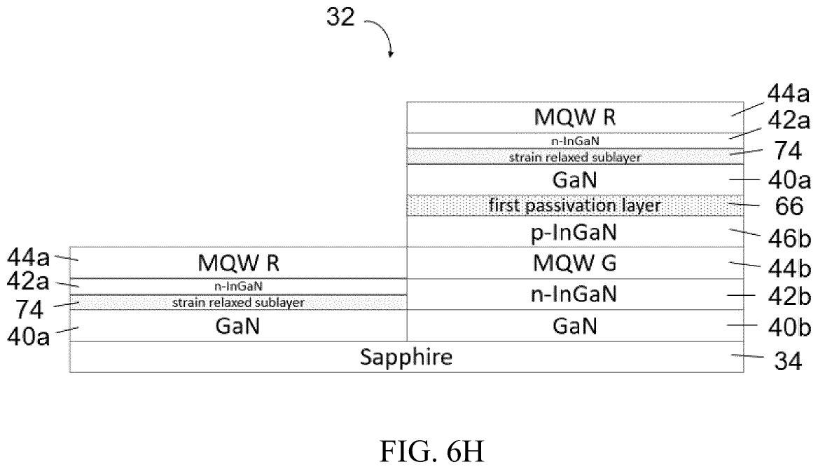

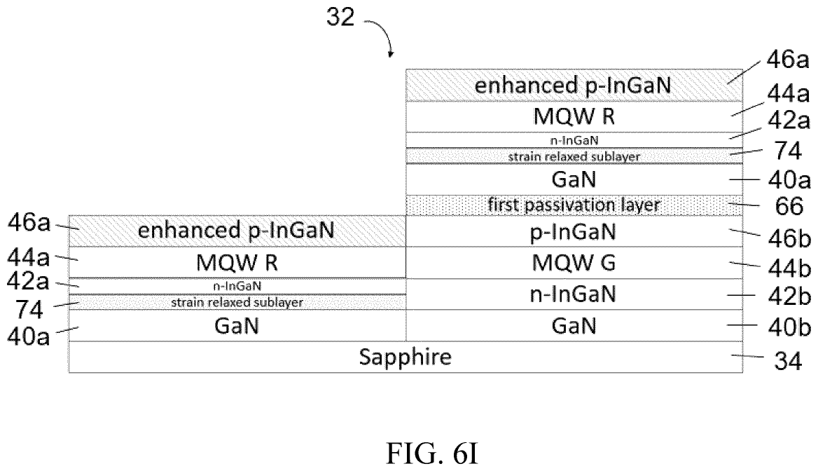

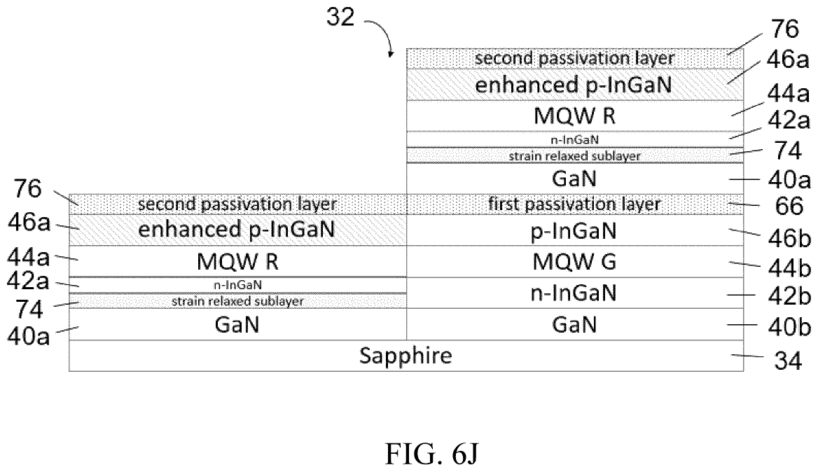

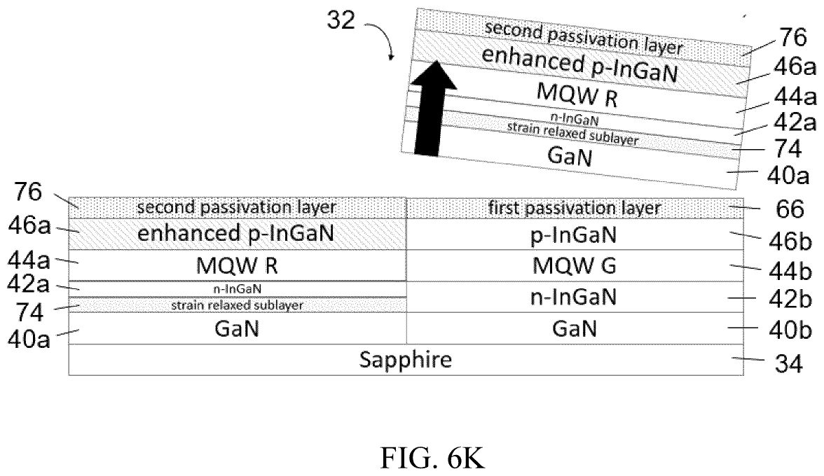

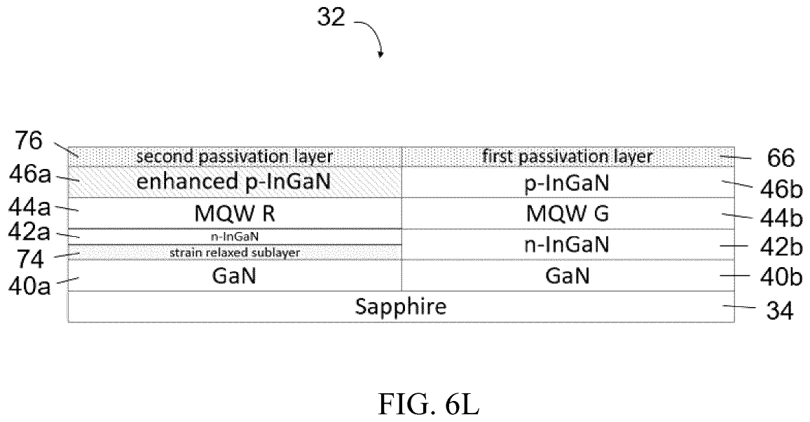

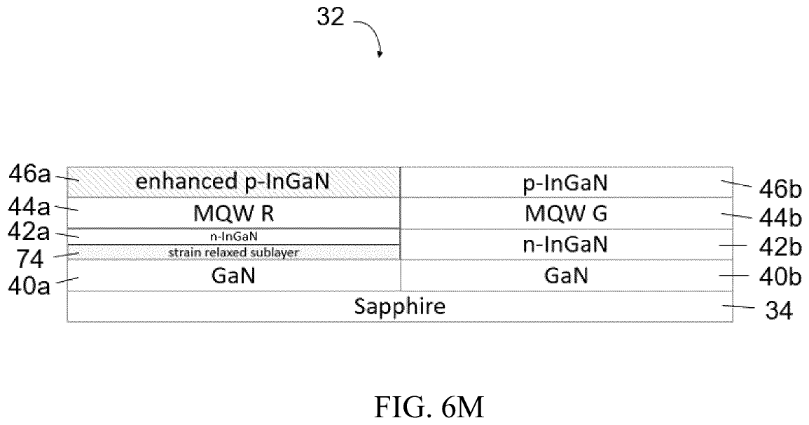

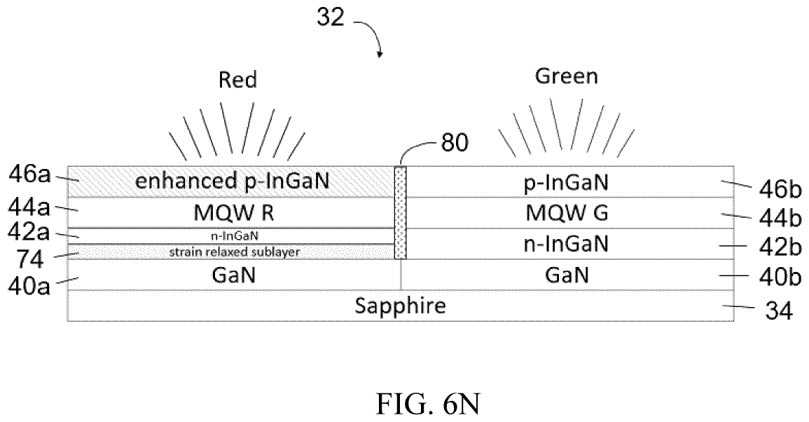

FIGS. 6A-6N provide an exemplary illustration of a process (Method A) to fabricate the optoelectronic structure 32 comprising a dual band light source based on red and green micro-LED subpixels, such as for use as a light source for an OCT. In this embodiment, the process starts with an as-grown green epitaxial wafer that comprises a sapphire substrate 34, upon which a buffer layer 40b of GaN is grown before depositing thereover, a green LED epitaxy stack of n-InGaN 42b, multi-quantum wells for green LED (MQW G) 44b, and p-InGaN 46b. Note that this green micro-LED wafer can be supplied in standard commercial form or custom made and tailored to a specific wavelength or waveform requirement of an OCT or hologram tool. Likewise, the exemplary red micro-LED epitaxy stack grown in this process is just one embodiment, which is shown for exemplary illustrative purposes. In at least some embodiments, an orange or yellow micro-LED light band may be substituted for red to provide a light band with a longer wavelength. In at least some embodiments, it is important to have reasonable separation in wavelength between the two light bands, for example a separation in the range of 30-50 nm may be preferred, while in other embodiments a separation of greater than 50 nm can be provided, such as at least a separation of 70-80 nm.

The process continues as shown in FIG. 6B by depositing a passivation layer 66, such as SiN or SiO₂. A photoresist (PR) material 68 is then deposited, and photolithographic techniques are used to remove the PR from those regions where the green subpixels are not intended, as shown in FIG. 6C. Using a semiconductor etching process, the undesired green epitaxial material is removed (see FIG. 6D). As shown, the entire green epitaxial stack, comprising p-InGaN, MQW G, n-InGaN, and GaN layers is removed down to the sapphire substrate 34 in the red region (alternatively, in some other embodiments, only the p-InGaN, MQW G, and n-InGaN layers would be etched away, thereby leaving the GaN surface intact and exposed). After the PR material 68 is removed (FIG. 6E), the wafer is now ready for further epitaxial growth deposition.

In FIG. 6F, within both red and green regions, a GaN layer 40a is deposited on the first passivation layer 66 and the sapphire, followed by a sublayer 72 that is deposited over the GaN layer. Note that the first passivation layer, deposited in FIG. 6B, exists upon the green region, protecting and shielding the green epitaxial stacks from the further deposition steps. The subsequent epitaxial layers grown on top of the first passivation layer 66 in these green regions are generally expected to be of poor quality, of little use, and will ultimately be removed. The sublayer 72 is then etched (FIG. 6G) to provide strain relaxation (now referred to as a strain relaxation layer 74 for clarity). One exemplary method for achieving strain relaxation is electrochemical (EC) porosification, which is known in the industry. A red epitaxial LED stack, comprising an n-InGaN layer 42a, a multi-quantum wells stack emitting red light (MQW R) 44a, and an enhanced p-InGaN layer 46a are then deposited as shown in FIGS. 6H-6I. Alternatively, in at least some embodiments, via selective regrowth or other growth conditions, the LED stack may only be grown in the red region with materials not deposited on the first passivation layer in the green region.

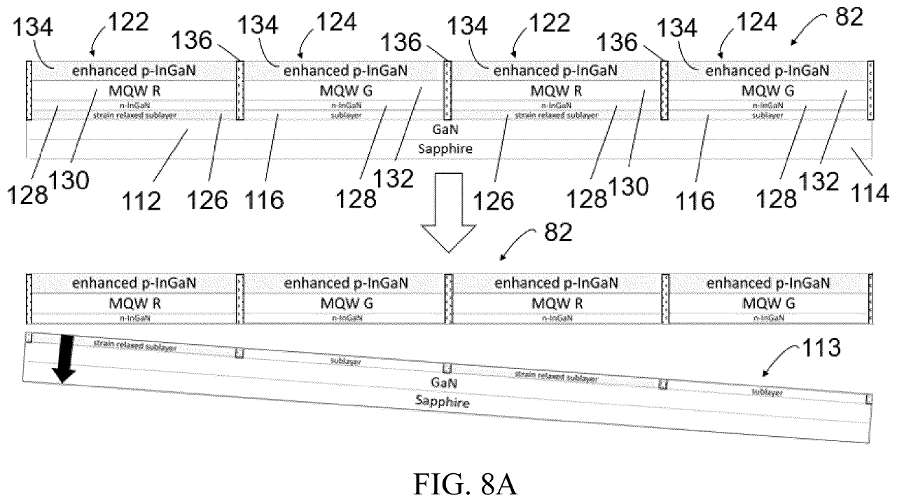

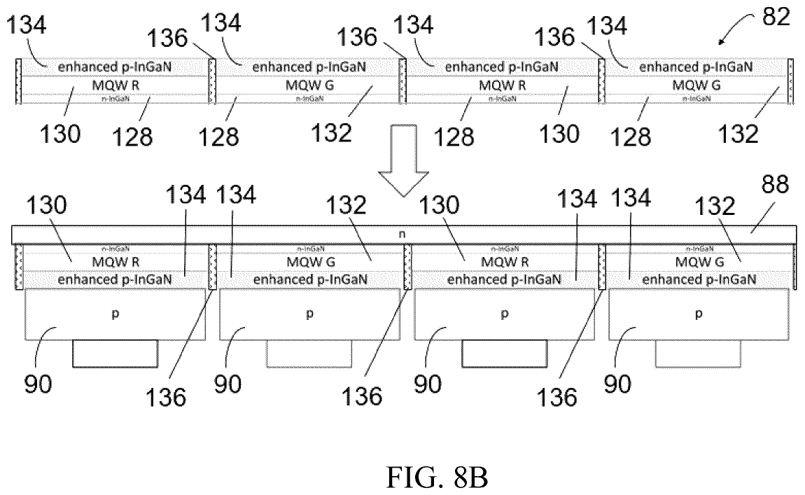

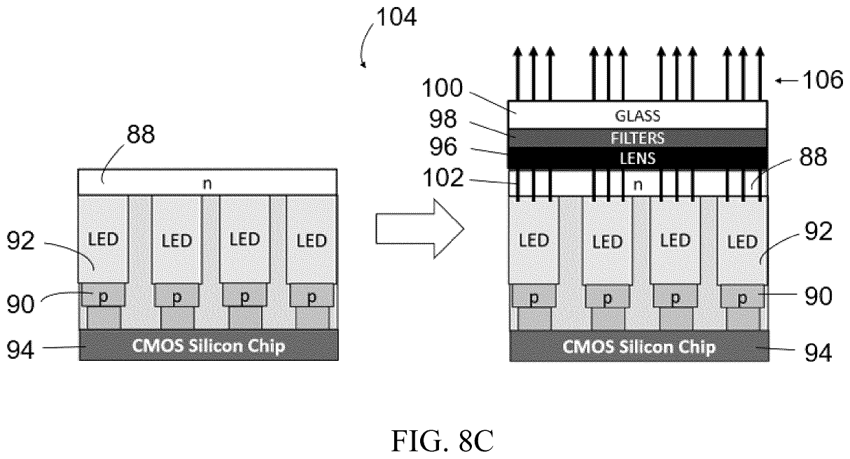

The process continues with a second passivation layer 76 being deposited to protect the red region (see FIG. 6J), and the amorphous epitaxial material deposited on the first passivation layer 66 in the green region is then removed as shown in FIGS. 6K and 6L. Next, the first and second passivation layers 66, 76 are removed as shown in FIG. 6M. An isolation sidewall 80 is then formed between the red and green epitaxial stacks via trench etching and oxide back-fill as seen in FIG. 6N. Optimized trench etching and passivation (via both commonly known methods as well as later discovered methods) and protection of sidewalls are utilized to maximize quantum efficiency and device performance of the LED stacks. In at least some embodiments, at this stage the optoelectronic structure is ready for additional processing, such as the removal of unnecessary lower elements, and the addition of a cathode and anodes, etc. One such exemplary process is shown in FIGS. 8A-8C and discussed below with regard to Method B.

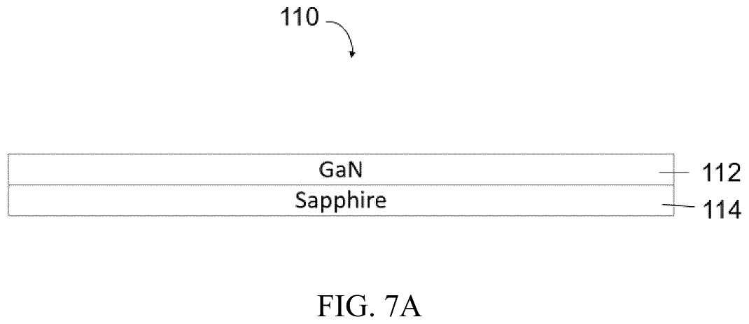

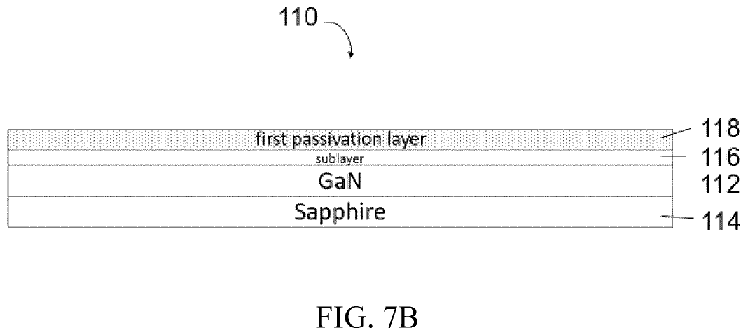

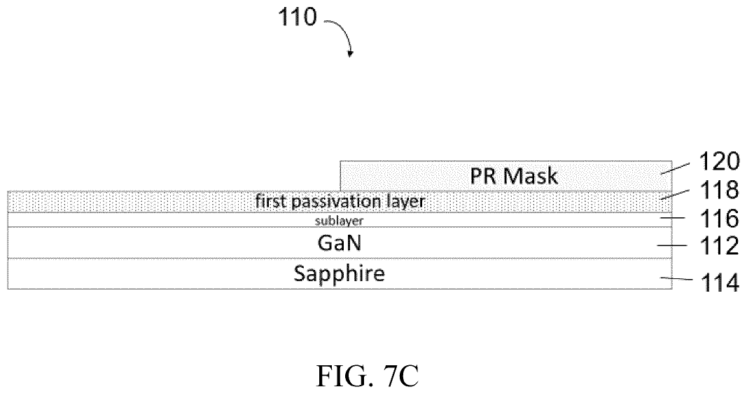

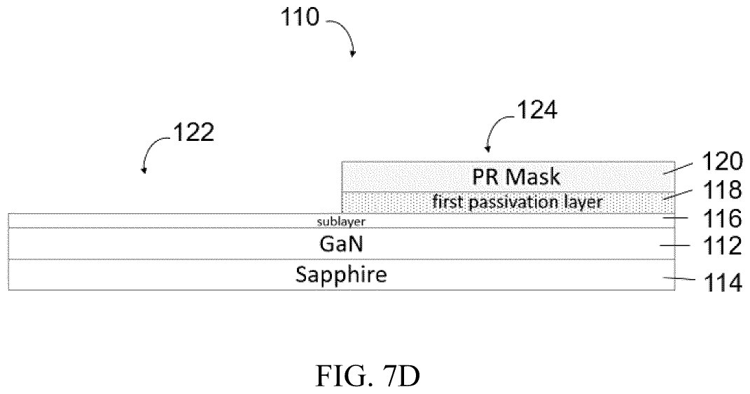

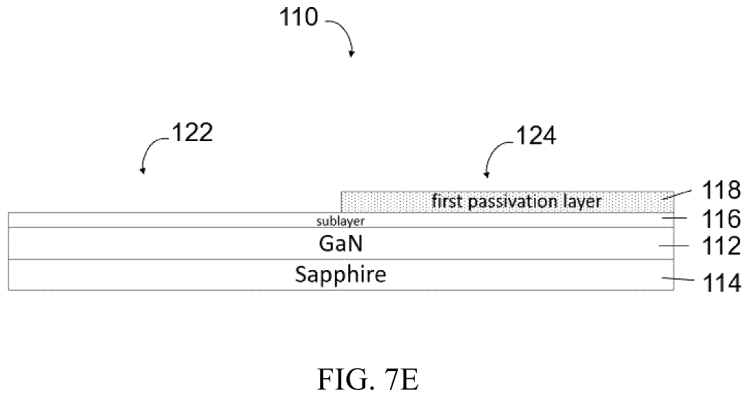

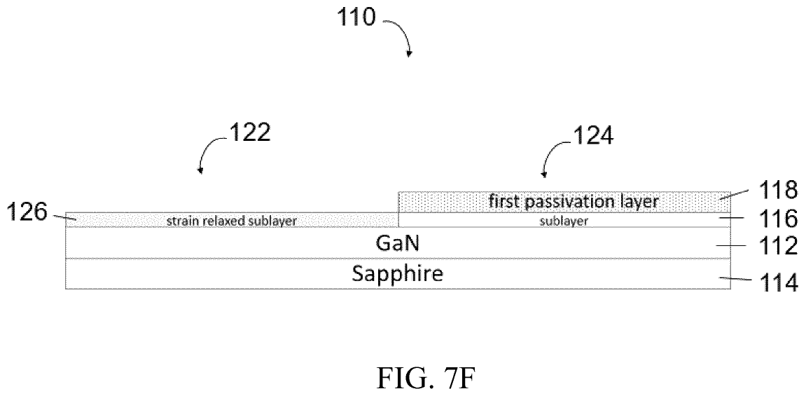

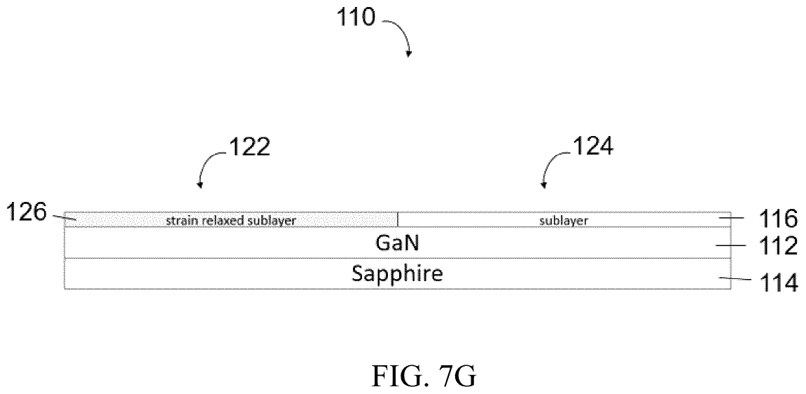

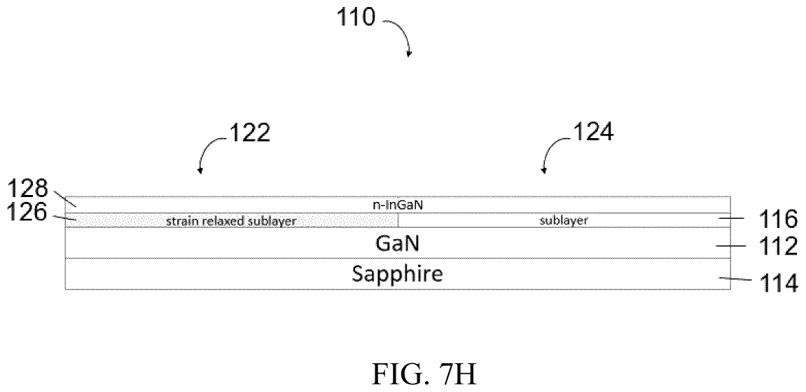

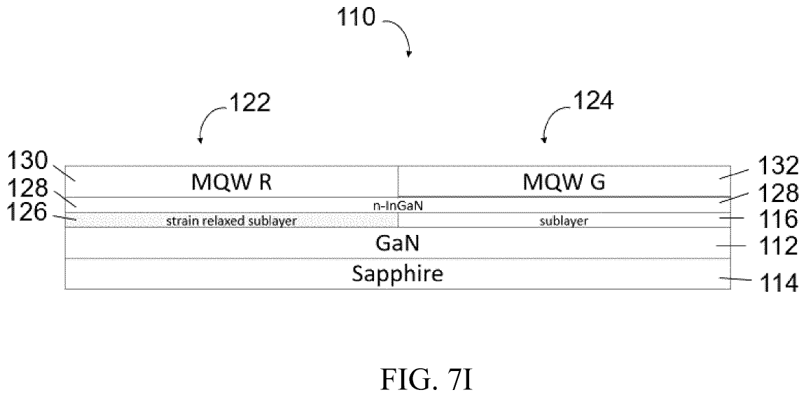

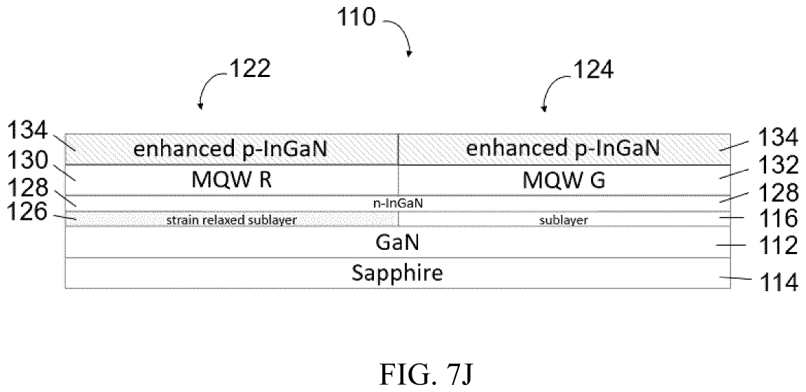

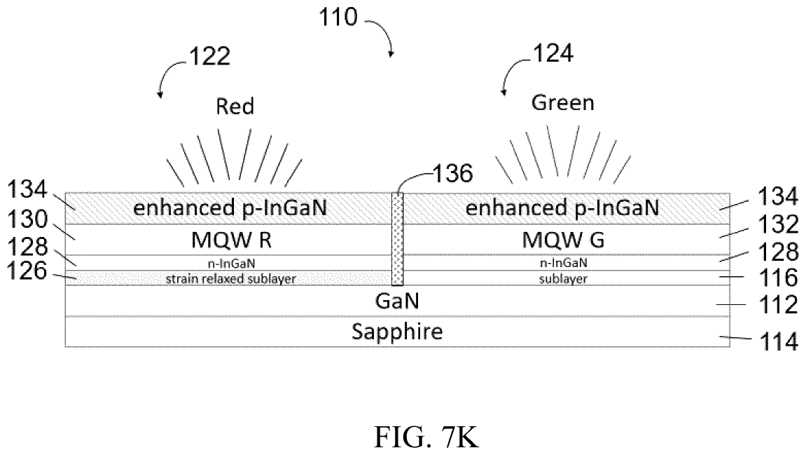

Referring now to FIGS. 7A-7K, illustrate another exemplary process (Method B) to fabricate an optoelectronic structure 110 comprising a dual band micro-LED, such as for use as a light source for an OCT device. The process can be in part or substantially duplicative of Method A in various ways using similar materials. Such material details are not all repeated here; although identical names may be used, for reference purposes, new element numbers have been provided in the drawings. To begin producing the optoelectronic structure 110, a buffer layer 112 comprising GaN is grown on a sapphire substrate 114 as shown in FIG. 7A, followed by depositing a sublayer 116 and passivation layer 118 as shown in FIG. 7B. A photoresist (PR) material 120 is then applied to mask the green region 122 (see FIG. 7C), and photolithographic techniques are used to remove the first passivation layer 118, exposing the sublayer 116 within the red region 122 as shown in FIG. 7D. After removing the photoresist (PR) material 120, as shown in FIG. 7E, electrochemical porosification or another technique is applied to the sublayer 116 in the red region 122 to provide strain relaxation (now referenced as strain relaxed sublayer 126 for clarity), while the sublayer 116 in the green region 124 is shielded by the passivation layer 118 (see FIG. 7F). The first passivation layer 118 over the green region 124 is then removed as shown in FIG. 7G. As a result, a substrate, comprising porous and non-porous sublayers (i.e. sublayer 116 and strain relaxed sublayer 126) is formed and ready for further processing. Through MOCVD or similar known techniques, InGaN epitaxy LED stacks, comprising n-InGaN 128, MQWs (e.g., MQW R 130, MQW G 132, or other desired MQWs) and enhanced p-InGaN 134, are grown on both the red and green regions 122, 124 in “one pass” (as shown in FIGS. 7H-7J). Because of the strain relaxation nature over the strain relaxed sublayer 126 in the red region 122, the LED epitaxy stack grown there will exhibit red shift towards longer wavelength, wherein the LED epitaxy stack grown over the sublayer 116 in the green region 124 will show no wavelength shift. The process then continues with the creation of an isolation sidewall 136 between the red and green epitaxial stacks via trench etching and oxide back-fill as illustrated in FIG. 7K. In at least some embodiments, at this stage the optoelectronic structure 110 is ready for additional processing, such as the removal of unnecessary lower elements, and the addition of a cathode and anodes, etc., as discussed below. The optoelectronic structure 110 can now be prepared for further packaging as a light source in various ways, such as described below with reference to FIGS. 8A-8C.

FIGS. 8A-8C illustrate exemplary further processing of an optoelectronic structure 82 (e.g., 32, 110, etc.). FIG. 8A shows an exemplary optoelectronic structure 82 that was formed using the method described above, but on a larger scale over a common sapphire substrate 84 and a common buffer layer 86 of GaN materials. Although only four epitaxial stacks having two red regions and two green regions alternating sequentially are shown for exemplary purposes, it shall be understood that in a typical manufacturing process the optoelectronic structure 82 can include a substantial number of epitaxial stacks (e.g., red and green, or other color regions) linearly or in an array on the common sapphire substrate 84 that are utilized to form pixels. For example, on a 6-inch wafer, there could be about 15,000+ pixels of 10 micron each from one end of the wafer to the other, so over 30,000 epitaxial stacks being fabricated.

As shown in FIG. 8A, the additional processing of the optoelectronic structure 82 includes the removal (e.g. lifted off) of a bottom portion 113 comprising the extraneous bottom levels that were utilized for manufacturing, but are not required to form the LEDs, leaving behind the epitaxy LED stacks of the red and green regions. More particularly, the base substrate 114, the buffer layer 112, and sublayers (e.g., strain relaxed sublayer 126 and sublayer 116), as well as any other additional layers that may have been utilized during manufacturing that are unnecessary for the final product, are removed.

As shown in FIG. 8B, the optoelectronic structure 82 can then be flipped (the process of flipping the polarities, or flip chip, is carried out before electric (e.g., ohmic) contacts are applied). A common cathode or anode bus can be constructed across subpixels or pixels to reduce the number of electrical interconnects. For example, as shown a common n-type layer is then deposited to form a cathode bus 88 on the top and a p-type layer is separately deposited on each of the epitaxial stacks to provide individual anodes 90 for the LEDs 92. Referring to FIG. 8C, the resulting structure is integrated with the CMOS or circuitry based on silicon or other materials (e.g., a CMOS chip 94) coupled to the anodes 90 to provide selective control of the LEDs 92. Next, finishing and packaging steps include incorporating additional elements, such as a lens 96, filter(s) 98, and glass layer 100 can added over the cathode bus 88 to filter and/or direct the emitted light 102 from the LEDs 92 and to protect them, thereby forming at least in part, a packaged micro-LED module/panel 104 with a dual band of red and green light emissions 106.

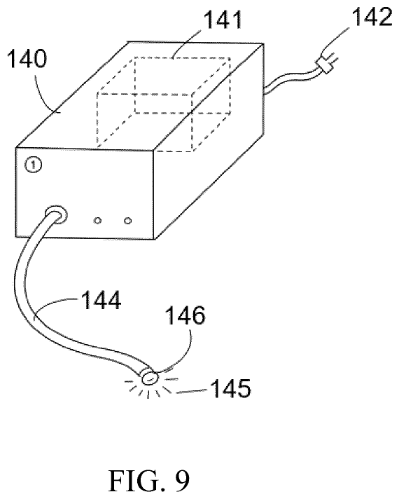

The packaged red and green micro-LED module/panel 104 (i.e. light source), or other variants, can be incorporated into various form factors including as a bench top OCT (see FIG. 9) with a housing 140 that encloses a light engine 141 that receives power (such as from a power cord 142, or other source), and comprises the micro-LED module/panel 104 (or another configuration fabricated from an optoelectronic structure, such as provided herein), which generates and conditions light used to create a scan, and which is coupled to wave guide/optical fibers 144 for emitting light 145 from an end portion 146. It shall be understood that in addition to the light source, the light engine 141 can include various commonly used components known in the art for OCT devices, such as wavelength control components, optical isolators, power stabilization and control electronics, temperature controls, etc. As such, the wavelengths of the emission bands from the light engine 141 can be tuned and calibrated, and their intensities can be altered via a control knob or dial by adjusting the driving current. Single beams, alternating beams, and/or pulsing or strobe light bands can be programmed and applied based on the morphological and physiological needs of the imaging and diagnosis applications. Omni-directionality and low-loss optical interconnect can be enabled by enclosing the MQW layers or action region in a 360-degree annular configuration using a distributed Bragg reflector (DBR) tailored to emission wavelengths of the subpixels. Referring again to FIG. 1, it shall be understood that an exemplary embodiment of an OCT (e.g. OCT system 10) may comprise a coupler/beam splitter 12, a display 14, a reference arm 16 directed to a mirror 18, a probed sample arm 20 directed to tissue 22, and a dual band micro-LED light source 24 that comprises the packaged red and green micro-LED module/panel 104, or other optoelectronic structure variants processed for such purposes.

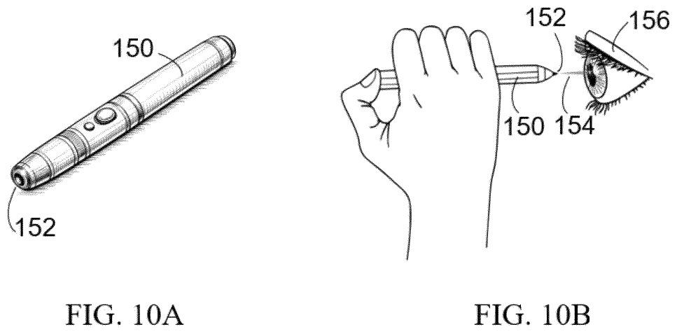

In at least some embodiments, the dual band or multi-band micro-LED light source fabricated using the aforementioned methods, can be incorporated into a handheld OCT device, such as a pen light 150 (pen-style form factor), as shown in FIG. 10A, for ease of carry and use. The pen light 150 can output red and green light beams 154 (or other wavelengths as desired) and collect backscattered light from a tip portion 152 to elucidate tissue form and thickness, as well as to enable early detection of infection, as shown in FIG. 10B examining an eye 156 Other colors or combinations of colors, including red, green and blue (RGB), can be used to construct such a light source. When equipped with requisite software, firmware, and hardware, this exemplary device can be used as a light source and a probe, wherein a medium-resolution or high-resolution camera is incorporated into the light source (or light engine). This device may comprise other peripheral features and functionalities including a battery charger, a communication device (e.g., Bluetooth transceiver, NFC transceiver, Wi-Fi chip, etc.), and a memory device (Flash memory chip, Micro-SD card, etc.) to ensure portability, interconnectivity, storage of recorded images, and streaming of videos while inspecting and carrying out diagnosis of eye tissues.

It is specifically intended that the invention not be limited to the embodiments and illustrations contained herein, but include modified forms of those embodiments including portions of the embodiments and combinations of elements of different embodiments. Moreover, it shall be understood that the various steps of the processes described and shown herein are exemplary, and in at least some embodiments can be modified to omit or add steps, as well as performed in a varied order. Additionally, although LEDs are discussed herein in detail, the optoelectronic structures should not be limited to LEDs, as the optoelectronic structures disclosed herein can include or be adapted to include LCD's or other light source configurations.

We claim:

1. An optoelectronic structure comprising, at least two light emitting diode (LED) subpixels, laterally, vertically, or any combination thereof arranged in a substantially common plane, wherein at least one LED subpixel emits light at a wavelength different from at least one other subpixel, and wherein the optoelectronic structure further includes at least: a buffer layer, a sublayer, or both; an n-doped III-nitride layer; a light emitting active region; and a p-doped III-nitride layer, wherein at least one of the at least two LED subpixels includes either a strain-relaxed buffer layer or strain-relaxed sublayer, or an enhanced p-doped III-nitride layer modified via dopant concentration adjustments, material composition changes, a porous structure, reduced thicknesses, or any combination thereof.

2. A method of manufacturing micro-LED subpixels that includes the at least two LED subpixels of claim 1, wherein the at least two LED subpixels may be laterally arranged, vertically arranged, or any combination thereof and monolithically fabricated on a substantially common plane on a structure wherein two or more light bands are emitted without requiring pick-and-place or mechanical transfer of subpixels.

3. The optoelectronic structure of claim 1, wherein the at least two micro-LED subpixels serve as a light source for use in optical imaging to illuminate a target object and to generate a multi-wavelength image of the target object.

4. The optoelectronic structure of claim 1, wherein the strain relaxation is achieved via electrochemical etching and porosification of a portion of the III-nitride buffer layer or sublayer, or both, within the region of an LED subpixel that emits light at a wavelength substantially different from at least one other subpixel on the substrate.

5. The optoelectronic structure of claim 1, wherein the strain relaxation can be achieved by V-defects, inclined threading dislocations, Si impurity doping, heteroepitaxy of different alloy compositions, other crystal defects, or any combination thereof.

6. The optoelectronic structure of claims 4 or 5, wherein the strain-relaxed region has a wurtzite in-plane lattice parameter between 99.5% and 100.5% of the in-plane wurtzite lattice parameter of at least one other region, preferably between 98% and 102%, and most preferably between 90% and 110%.

7. The optoelectronic structure of claims 4 or 5, wherein the strain-relaxed region has a wurtzite in-plane lattice parameter, α, larger than GaN, at least one adjacent layer, or any combination thereof.

8. The optoelectronic structure of claim 1, wherein the strain relaxation buffer layer is at least partially UID.

9. The optoelectronic structure of claim 1, wherein the strain relaxation buffer layer is at least partially n-type.

10. The optoelectronic structure of claim 1, wherein the strain relaxation buffer layer is at least partially p-type.

11. The optoelectronic structure of claim 1, wherein the strain relaxation buffer layer is at least partially relaxed.

12. The optoelectronic structure of claim 1, wherein the strain relaxation buffer layer is at least fully relaxed.

13. The optoelectronic structure of claim 1, wherein the enhancement of the p-doped III-nitride layer is achieved by modifying the material composition and/or dopant concentration within at least a portion of the p-layer and/or across the interface with the p-layer, wherein the variation may not be limited to a smooth or monotonic gradient, for the LED subpixel that emits light at a wavelength longer than at least one other subpixel on the substrate.

14. The optoelectronic structure of claim 1, wherein the enhancement of the p-doped III-nitride layer is achieved via etching and porosification of an extra layer adjacent or in proximity to the p-layer for the LED subpixel that emits light at a wavelength longer than at least one other subpixel on the substrate.

15. The optoelectronic structure of claim 1, wherein at least one of the LED subpixels has at least one p-type layer with a thickness less than 100 nm; preferably less than 50 nm; and most preferably less than 10 nm.

16. The optoelectronic structure of claim 1, wherein at least one of the LED subpixels is activated in situ.

17. The optoelectronic structure of claim 1, wherein at least one of the LED subpixels contains at least one tunnel junction layer or sublayer.

18. The optoelectronic structure of claim 17, wherein significant enhancements in light directionality of dual or multi bands and their subpixel size reduction are achieved via at least one tunnel junction layer of sublayer in a stacked or cascaded monolithic RG or RGB micro-LED device.

19. The optoelectronic structure of claim 17, wherein significant enhancements in current spreading and/or reduction in optical loss within the light source or optical fiber interconnect are achieved via at least one tunnel junction layer.

20. The optoelectronic structure of claim 1, wherein at least one of the LED subpixels contains at least one n-type layer or sublayer on or above at least one p-type layer or sublayer.

21. The optoelectronic structure of claim 1, wherein at least one of the LED subpixels may undergo at least one flip-chip process.

22. The optoelectronic structure of claim 1, wherein the III-nitride layers include a buffer layer, strain-relaxed sublayer, n-doped III-nitride layer, active region, and the enhanced p-doped III-nitride layer can be comprised of any alloy composition of the (La, Y, Sc, Ga, Al, In, Th, B)N semiconductors having the formula LaₛYₜScᵤGaᵥAl_{w}InₓTh_{y}B_{z}N where 0≤s≤1, 0≤t≤1, 0≤u≤1, 0≤v≤1, 0≤w≤1, 0≤x≤1, 0≤y≤1, 0≤z≤1, and s+t+u+v+w+x+y+z=1.

23. The optoelectronic structure of claim 9, wherein the III-nitride layers include at least one III-N layer that contains at least 10¹⁷ cm⁻³ concentrations of Si, Ge, C, O, S, Be, Mg, Ca, Fe, or any combination thereof.

24. The optoelectronic structure of claim 1, wherein the difference of peak wavelengths between any two LED subpixels is between 30 and 300 nm when under forward electrical bias across the LED pixels; preferably between 50 and 150 nm; and most preferably between 80 and 120 nm.

25. The optoelectronic structure of claim 1, wherein the difference of peak wavelengths between any two LED subpixels is between 30 and 300 nm when under forward electrical bias across the LED pixels; preferably between 50 and 150 nm; and most preferably between 80 and 120 nm.

26. The optoelectronic structure of claim 1, wherein at least part of one light emitting active region of at least one LED subpixel has the same III-nitride composition of at least part of one light emitting active region of at least one other LED subpixel with different quantum well thicknesses, AlGaN cap composition and thicknesses.

27. The optoelectronic structure of claim 1, wherein at least part of one light emitting active region of at least one LED subpixel contains at least 15% Indium as the cation.

28. The optoelectronic structure of claim 1, wherein at least one quantum well in at least part of one light emitting active region of at least one LED subpixel is at least 3 nm thick.

29. The optoelectronic structure of claim 1, wherein an Aluminum composition is at least one layer disposed adjacent to at least one III-N light emitting active region in at least one LED subpixel is greater than 15%.

30. The optoelectronic structure of claim 1, wherein an Aluminum composition is at least one layer disposed adjacent to at least one III-N light emitting active region in at least one LED subpixel is greater than 25%.

31. The optoelectronic structure of claim 1, wherein at least one III-N light emitting active region contains at least 5e17 cm⁻³ concentrations of Si.

32. The optoelectronic structure of claim 1, wherein the difference in emission spectrum of different LED subpixels is at least partially due to difference in strain relaxation, alloy composition, or any combination thereof.

33. The optoelectronic structure of claim 1, with III-nitride LED subpixels, wherein at least one of the LED subpixels has an emitting wavelength centered at no less than 480 nm.

34. The optoelectronic structure of claim 13, wherein the process of p-layer enhancement comprises annealing of a p-type layer in the presence of a porous layer in proximity to and on top of it.

35. A light source comprising at least two micro-LED subpixels whose peak wavelengths differ by at least 30 nm from each other and are configured for use in Optical Coherence Tomography, holographic displays, endoscopy, ophthalmology, and/or other biomedical imaging and diagnosis tools to discern body tissue morphology, cell structures, blood cell existence, and/or vascular dynamics within blood vessels and/or structures within the retina or other human organs and tissues.

36. The light source of claim 35, wherein at least one LED subpixel is always emitting light, sometimes emitting light, never emitting light, or any combination thereof.

37. The light source of claim 35, wherein multiple different LED subpixels may emit light simultaneously, at different times, or any combination thereof during operation.

38. The light source of claim 35, wherein the optoelectronic structure's light emission pattern is directional, non-directional, omni directional, spherical, hemispherical, or any combination thereof.

39. The optoelectronic structure of claim 1, wherein a base substrate is provided to receive the buffer layer thereon, with the base substrate comprising sapphire, silicon, silicon carbide, gallium nitride, or aluminum nitride.

40. The optoelectronic structure of claim 1, wherein the structure is formed in a c-plane metal-polar, c-plane nitrogen polar, nonpolar, or semipolar orientation.

41. The optoelectronic structure of claim 1, wherein at least some photons from at least one light emitting region emitted from at least one excited state energy level of the structure, include e1h2, e2h1, e2h2, or e1h3, or any combination thereof.

42. A micro-LED device comprising:
a first LED subpixel that emits light at a first wavelength, and comprises:
at least one of a first buffer layer and a first sublayer;
a first n-doped III-nitride layer;
a first light emitting active region; and
a first p-doped III-nitride layer; and
a second LED subpixel that emits light at a second wavelength that is different than the first wavelength, and comprises:
at least one of a second buffer layer and a second sublayer; a second n-doped III-nitride layer;
a second light emitting active region; and
a second p-doped III-nitride layer;
wherein the first and second LED subpixels are laterally, vertically, or any combination thereof arranged on a substantially common plane, and wherein at least one of the first buffer layer, first sublayer, second buffer layer, and second sublayer is either strain-relaxed or is an enhanced p-doped III-nitride layer modified via at least one of dopant concentration adjustments, material composition changes, and a porous structure.

43. A method of manufacturing the micro-LED device of claim 42, wherein the first LED subpixel and second LED subpixel are micro-LED subpixels that are laterally or vertically arranged and monolithically fabricated on a substantially common plane on a structure without requiring pick-and-place or mechanical transfer of the subpixels.

44. A micro-LED device comprising an optical imaging device that includes multiple LED subpixels that are laterally, vertically, or any combination thereof arranged and monolithically fabricated on a substantially common plane on a structure without requiring pick-and-place or mechanical transfer of the subpixels as light sources, wherein the micro-LED subpixels are configured to emit in multiple emission spectra and illuminate a target object and to generate a multi-wavelength image of the target object.

45. A micro-LED device of claim 44, wherein the strain relaxation is achieved via V-defects, or electrochemical etching and porosification of a portion of the III-nitride buffer layer or sublayer, or both, and within the region of the first LED subpixel that emits light at a wavelength substantially different than the second LED subpixel.

46. A micro-LED device of claim 44, comprising the LED structure of claim 45 and an image sensor selected from a group consisting of a solid-state single-photon avalanche diode (SPAD) array, a quantum-dot-enhanced charge-coupled device (CCD) sensor, a quantum dot-enhanced complementary metal-oxide-semiconductor (CMOS) sensor, and a CCD or CMOS image sensor; and optionally a nonlinear optical element positioned to receive refracted light from the body tissue.

47. A micro-LED device of claim 44, wherein the optoelectronic structure can be configured and enhanced by enclosing the MQW layers (or action region) of the device in a 360-degree annular configuration using a distributed Bragg reflector (DBR) tailored to the emission wavelengths of the subpixel optimized for use in quantum photonics, quantum sensing, or artificial intelligence (AI) data communications applications requiring high modulation bandwidth and directionality control.

48. An Optical Coherence Tomography equipment or device, wherein intravascular OCT and endoscopic applications requiring the use of two or more light bands via an optical fiber to transmit light to the tissue through portable, insertable, or maneuverable forms and collect backscattered signals by insertion into coronary arteries and other intestines or ducts to detect inflammation or early cancers. These adaptations enable high-resolution “optical biopsy” but would require higher electroluminescence and efficient emission of visible light and low loss in optical interconnect.

49. A light source comprising at least two micro-LED subpixels whose peak wavelengths differ by at least 30 nm from each other having waveforms with a full-width at half-maximum (FWHM) of at least 10 nm and are utilized as a light source in a biomedical imaging device to discern at least one of body tissue morphology, cell structures, blood cell existence, vascular dynamics within blood vessels, structures within the retina or tissues within other human organs.

50. A light source of claim 49 for use in optical imaging comprising: at least two micro-LED subpixels as claimed in claim 1, wherein the at least two micro-LED subpixels are configured to illuminate a target object and to generate a multi-wavelength image of the target object.

51. An optical imaging device or equipment that includes as a light source the first subpixel and second subpixel as provided in claim 29, wherein the first subpixel and second subpixel are micro-LED subpixels are configured to illuminate a target object and to generate a multiwavelength image of the target object.

52. A light source for visible-light optical coherence tomography (vis-OCT), comprising: a monolithic micro-LED array comprising a plurality of InGaN-based micro-LED pixels, each pixel having a lateral dimension of less than 100 μm; wherein the micro-LED array is configured to emit light across a broadband visible spectrum including at least blue, green, and red wavelength bands, the emitted light having a low temporal coherence length suitable for achieving axial resolution of 2 μm or less in tissue and high spatial coherence for enhanced image sharpness with reduced speckle noise.

53. The light source of claim 52, wherein the micro-LED array comprises engineered V-defects in the epitaxial structure to enable localized carrier injection via semipolar sidewalls, thereby improving hole injection efficiency and screening of quantum-confined Stark effect (QCSE) in high-indium-content red-emitting quantum wells.

54. The light source of claim 52, wherein the micro-LED array is configured for programmable multi-band emission, with at least two independently addressable wavelength bands within the visible range, enabling spectroscopic vis-OCT or dual-modal imaging.

55. The light source of claim 52, wherein the InGaN-based micro-LED pixels include single, double or multi-layer V-defect structures to relieve compressive strain in red-emitting quantum wells, thereby increasing internal quantum efficiency (IQE) and enabling peak external quantum efficiency (EQE) greater than 5% at red wavelengths in pixels smaller than 50 μm.

56. The light source of claim 52, further comprising a meta surface integrated over the micro-LED array to enhance directionality and collimation of emitted light, improving coupling efficiency into optical fibers or probes for endoscopic or handheld vis-OCT applications.

57. The light source of claim 52, wherein the micro-LED array is fabricated using metalorganic chemical vapor deposition (MOCVD) with controlled low-temperature InGaN quantum well growth followed by higher-temperature barrier layers, incorporating superlattice pre-layers to nucleate V-defects for strain relaxation and improved indium incorporation.

58. The light source of claim 52, wherein the emitted light has a spectral bandwidth per band or across the array sufficient to achieve an axial resolution better than 1.5 μm in aqueous tissue, with speckle contrast reduced by at least 50% compared to narrowband laser sources.

59. The light source of claim 52, wherein the micro-LED pixels are passivated with atomic layer deposition (ALD) layers to reduce sidewall recombination, maintaining high EQE in pixels smaller than 20 μm for compact array integration.

60. The light source of claim 53, wherein the V-defects have a density in a range of 10⁸ to 10¹^{⁰} cm⁻² and lateral opening sizes between 150 nm and 400 nm, configured to promote three-dimensional current pathways and localized high carrier density for enhanced QCSE screening at low current densities.

61. The light source of claim 53, wherein the semipolar sidewalls of the V-defects comprise {10 11} facets, reducing polarization-induced barriers for hole transport and enabling radiative recombination primarily through lateral injection into c-plane quantum wells.

62. The light source of claim 54, wherein the independently addressable wavelength bands include a red band centered between 600 nm and 670 nm, and the micro-LED pixels in the red band utilize optimized InGaN quantum wells with graded composition and/or enhanced multiple quantum well designs to mitigate QCSE and achieve low temporal coherence.

63. The light source of claim 54 wherein the independently addressable wavelength bands include a red band centered between 600 nm and 670 nm, and the micro-LED pixels in the red band utilize optimized p-layer with graded composition or enhanced and/or ultrathin designs to mitigate QCSE and achieve low temporal coherence.

64. The light source of claim 54, wherein the drive circuitry enables pulse-mode operation with variable relative intensities across wavelength bands, supporting functional vis-OCT modalities such as retinal oximetry or multi-chromophore contrast imaging.

65. A light source of claim 55, wherein the single, double or multi-layer V-defect structures increase IQE by at least 100% compared to planar c-plane structures without V-defects, as measured at low current densities relevant to portable vis-OCT operation.

66. An Optical Coherence Tomography (OCT) illumination system, comprising:
a micro-LED array light source including a plurality of InGaN-based micro-LED pixels fabricated on a common substrate; drive circuitry coupled to the micro-LED array to selectively activate subsets of pixels for structured illumination; wherein the light source provides broadband emission with an effective coherence length configured to support sub-2 μm axial resolution in vis-OCT while minimizing speckle artifacts without requiring phase-randomization averaging techniques.

67. The OCT illumination system of claim 66, wherein the structured illumination comprises full-field, line-field, or multi-spot patterns generated by selective pixel activation, eliminating or reducing mechanical scanning requirements in the vis-OCT system.

68. A vis-OCT device, comprising:
the light source of claim 52;
an interferometer configured to split light from the light source into a sample arm and a reference arm; a detector configured to capture interference signals from backscattered light;
and processing circuitry configured to generate cross-sectional or three-dimensional images of tissue with micrometer-scale resolution.

69. A vis-OCT device of claim 68, wherein the micro-LED array is integrated into a handheld probe or endoscopic catheter, the device being portable and battery-powered, and configured for point-of-care imaging in ophthalmology, dermatology, or intravascular applications.

70. A vis-OCT device of claim 68, further comprising fiber-optic coupling between the micro-LED array and the sample arm, wherein the high spatial coherence of the micro-LED emission enables efficient single-mode or few-mode fiber delivery with minimal loss.

71. A vis-OCT device of claim 69, configured for visible-light spectroscopic imaging, wherein the multi-band emission from the micro-LED array enables simultaneous structural OCT and absorption-based contrast for early detection of tissue abnormalities, such as microvascular changes or chromophore distributions.
